(19) United States
(12) Patent Application Publication
(10) Pub. No.: US 2015/0037207 A1
(43) Pub. Date: Feb. 5, 2015

(54) MODULAR FORKLIFT SANITIZATION TROUGH

(71) Applicants: ANTHONY JOSEPH TERRANOVA, LAGUNA HILLS, CA (US); JEFFREY ROY HENRY, BALLSTON LAKE, NY (US)

(72) Inventors: ANTHONY JOSEPH TERRANOVA, LAGUNA HILLS, CA (US); JEFFREY ROY HENRY, BALLSTON LAKE, NY (US)

(21) Appl. No.: 14/447,606

(22) Filed: Jul. 30, 2014

Related U.S. Application Data
(60) Provisional application No. 61/859,775, filed on Jul. 30, 2013.

(51) Int. Cl. A61L 2/18

(52) U.S. Cl. CPC A61L 2/18
USPC 422/28; 422/292

(57) ABSTRACT

The present inventive subject matter is drawn to systems and methods of disinfecting and sanitizing the tires and forks of industrial forklifts. In one aspect of this invention, a modular forklift disinfecting system is presented comprising a sanitizing trough designed to allow driving a forklift up a first ramp, into the sanitizing trough containing a sanitizing solution, and out the other end of the sanitizing trough down a second ramp. The system is designed to operate without diverting the forklift from its normal business operation, or utilizing any extra machinery. In another aspect of this invention, a method for disinfecting the tires and forks of industrial forklifts is also presented.

### RELATED APPLICATION(S)

This application is based on and takes priority from Provisional Application Ser. No. 61/859,775, filed on Jul. 30, 2013, entitled “MODULAR FORKLIFT SANITIZATION TROUGH,” which is incorporated by reference herein.

### FIELD OF THE INVENTION

The present invention relates generally to disinfecting and sanitizing industrial equipment and machinery operated by the food processing industry and similar use markets. In particular, the present invention relates to the field of cleaning and sanitizing industrial forklifts used in food production and packaging facilities.

### BACKGROUND OF THE INVENTION

Control of microbiological contamination is of major concern in all food processing and packaging facilities. Listeria, salmonella and e-coli are but only a few of the food-borne pathogens that can infect our food sources and cause illness and death. Exhaustive studies to determine how most infections entered a food production facility revealed that one of the major sources of food plant contamination were the tires and forks of forklifts. Swab testing indicated a high degree of microbiological infection occurred when the forklifts off-loaded delivery trucks and when the forklifts were driven out of doors and allowed to re-enter the processing or warehousing areas unsanitized. Most food plants designate forklifts essentially “inside lifts” transferring inventory from box trucks to the plant. This was a major source of microbiological contamination especially if the truck is not washed on a regular basis. Freight carriers seldom if ever are required to sanitize their equipment and often times deliver to numerous different facilities in different industries, potentially spreading spoilage and illness-causing microorganisms.

Other forklifts may be “outside” lifts, i.e., they are generally used outside of a facility, where both the tires and the forks (also known as blades or tines) may be highly contaminated with dirt and infectious organisms. Some methods of sanitation that are viable to a certain extent include doorway foaming systems, which are the primary method currently employed to sanitize forklift tires. These systems spray a layer of sanitizing foam across doorways where forklifts enter or exit warehousing or processing areas. These systems use high-foaming surfactants or quaternary compounds that cover the floors with several inches of thick foam. The forklifts drive through the foam that adheres to the tires and, in theory, sanitizes them. Other approaches include disinfectant mats soaked with disinfectant, over which a forklift passes when entering a facility, thus squelching disinfectant over the tires, which disinfectant soaks back into the mat once the forklift has passed. Other than foaming systems and disinfectant mats, steam cleaning requires that the forklift be kept out of operation while the cleaning process takes place.

These systems have several inherent problems. First, the foam may be transported on the tires several feet beyond the doorway thereby causing hazardous working environments. Secondly, if the forks are too low and allowed to drag through the foam layer, the product being transported can be contaminated with the sanitizing foam.

There is a need for a solution to disinfect the forks and tires of forklifts, requiring minimum extra time and effort, for use in the food processing industry and similar use markets. A design to address this need is hereby presented.

### BRIEF DESCRIPTION OF THE FIGURES

FIG. 1 illustrates an example of an embodiment of a modular forklift disinfecting system in accordance with the invention, comprising two ramp sections each having a length of approximately twenty four inches, and a trough section having a length of approximately seventy two inches.

FIG. 2 illustrates another example of an embodiment of the modular forklift disinfecting system in accordance with the invention, comprising two ramp sections each having a length of approximately eighteen inches, and a trough section having a length of approximately eighty four inches.

FIG. 3 illustrates the connecting hardware as it is inserted and fastened within depressed cutout areas located within each of the ramp sections and the troughs of the modular forklift disinfecting systems shown in FIGS. 1 and 2.

FIG. 4 illustrates a trough section having two depressed cutout areas including connecting means that may be inserted and fastened within each of the depressed cutout areas.

FIG. 5 illustrates an example of yet another embodiment of the modular forklift disinfecting system in accordance with the invention, comprising a trough section having a single trough.

FIG. 6 illustrates a flow chart of a method for disinfecting the tires and forks of industrial forklifts using a modular forklift disinfecting system.

### BRIEF SUMMARY OF THE INVENTION

The present inventive subject matter is drawn to systems and methods to disinfect and sanitize the tires and forks of industrial forklifts. In one aspect of this invention, a modular forklift disinfecting system is presented. In some embodiments, the modular forklift disinfecting system may comprise a first ramp section, a trough section configured to contain a sanitization solution and having a first end and a second end, and a second ramp section. The first ramp section may be removably connected to the first end of the trough section and the second ramp section may be removably connected to the second end of the trough section. In examples of other embodiments, the trough section may include a plurality of trough sections, for example, a first trough section and a second trough section.

In an embodiment having two trough sections, each of the first trough section and the second trough section may have an inner rail and an outer rail. The outer rail may be higher in height than the inner rail. In an example of an embodiment, the first rail section may have a height of approximately three and one quarter inches. In other embodiments, the second rail section may lower in height than the first rail section by three eighths of an inch. The first rail section and the second rail section may have a width of two inches in some other embodiments. Also, in yet another embodiment, the first rail section and the second rail section may be half-trapezoid-shaped walls.

In examples of other embodiments, the first ramp section, the second ramp section and the trough section may be connected together by way of inserting a connecting means into one or more wall depression areas located within the walls of the different sections. In some embodiments, each of the first and second ramp sections may have a length between eighteen to twenty four inches, and may have a width of approximately seventy two inches.

In examples of other embodiments, each trough may have two ramp areas, where each ramp area may have a slope from a height of three and one quarter inches to a height of one half of an inch. Each trough may also have a length between seventy two to eighty four inches, and the troughs may have a combined a width of approximately seventy two inches. Each trough may include a floor section having a length of approximately thirty four inches, in other embodiments. The floor section may have a length of approximately forty four inches.

In examples of other embodiments, the modular forklift disinfecting system may comprise a base plate placed under the first ramp section, the second ramp section, and the trough section. The base plate may be made of rubber material and have a height of three quarters of an inch. In each of these embodiments, the sanitizing solution may be selected from a group consisting of quaternary ammonium chloride, chlorine dioxide, peracetic acid, acid anionic, bleach, and an isopropyl alcohol-based sanitizer.

In another aspect of this invention, a method for disinfecting the tires and forks of industrial forklifts is presented. In some embodiments, the method of disinfecting the tires and forks of industrial forklifts comprises the steps of (i) arranging one or more troughs at a middle section of an operating area; (ii) arranging a first ramp section at a first end of the one or more troughs; and (iii) arranging a second ramp section at a second end of the one or more troughs where the second end is at the opposite end to the first end of the one or more troughs. The method may further comprise the step of filling the one or more troughs with a sanitizing solution. The method may also comprise the steps of (i) driving a forklift up the first ramp section; (ii) driving the forklift forward until the wheel set of the forklift is in the one or trough sections; (iii) driving the forklift forward to ensure the remaining areas of the wheel set are immersed in the sanitizing solution; and (iv) driving the forklift forward out of the one or more troughs and down the second ramp section.

In examples of some methods, the method may further comprise the steps of (i) lowering the forks of the forklift until immersed in the sanitizing solution in the one or more troughs; and (ii) lifting up the forks of the forklift out of the sanitizing solution after a predetermined time period of immersion. In each of these methods, the sanitizing solution is selected from a group consisting of quaternary ammonium chloride, chlorine dioxide, peracetic acid, acid anionic, bleach, and an isopropyl alcohol-based sanitizer.

### DETAILED DESCRIPTION

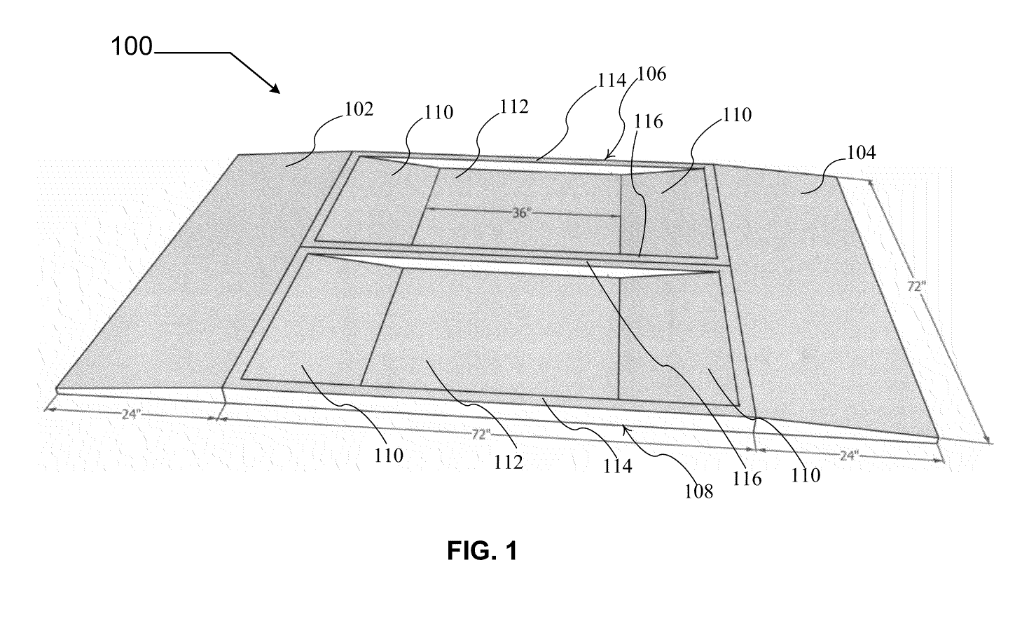

The present inventive subject matter is drawn to systems and methods for disinfecting and sanitizing the tires and forks of industrial forklifts In one aspect of this invention, a modular forklift disinfecting system is presented. The design of the system described herein allows for the sanitization of the tires and forks of a forklift without diverting it from its normal business operation, or utilizing any extra machinery. More specifically, the forklift disinfecting system comprises a sanitizing trough designed to allow driving a forklift up a ramp into the trough containing a sanitizing solution, and out the other end down a second ramp. FIG. 1 shows a simplified embodiment of such a system.

As shown in FIG. 1, the modular forklift disinfecting system 100 may include a first ramp section 102, a second ramp section 104, and a trough section 106 108. As shown, the trough section may include a first trough 106 and a second trough 108. The first 102 and second 104 ramp sections are arranged at opposite ends of the system, while the first 106 and second 108 trough sections are arranged adjacent to each other at the middle area of the system, separating the first 102 and second 104 ramp sections. The first 106 and second 108 trough sections may be configured to contain a sanitizing solution for rinsing and disinfecting the tires and/or the forks of the forklift. Each of the first 106 and second 108 trough sections may be designed to have two opposite sloping surfaces (trough ramp sections 110) that allow the tires of a forklift to descend inside, and to ascend making its way outside of the trough. Each trough section may also include a floor section 112. The forklift may travel up the first ramp section 102, then back down into the trough's floor section 112 containing the sanitizing solution. After traveling through the solution, the forklift travels back up an outgoing ramp 110, down the second ramp section 104, and the tires would thereby emerge disinfected. In some embodiments, the trough sections 106 108 may be at a 3¼ height sloping down to a ½ height, which provides a depth of approximately 2 inches for the containment of a suitable amount of the sanitizing solution while offering a smooth and gradual travel path for the forklift.

As such, the system design allows the sanitizing solution to be contained within each of the troughs, thereby minimizing the risk of contaminating any transported product. The forks of the forklift may also be sanitized as they are dropped into the sanitizing trough, preferably for thirty to sixty seconds. The system as designed is modular and mobile and can be placed anywhere within a working facility, where forklift traffic is centered. The sanitizing solution may be selected from a group consisting of quaternary ammonium chloride, chlorine dioxide, peracetic acid, acid anionic, bleach or isopropyl alcohol-based sanitizers. In a preferred embodiment, metal corrosivity and other typical considerations may be considered in selecting the sanitizing solution in order to minimize the risk of damaging the forklift or other operating machinery.

In other embodiments, the first 106 and second 108 trough sections may include a rail section on each of their sides. The first rail section 114 may be facing outward of the system, while the second rail section 116 may be facing inward and abut the second rail section 116 of the adjacent trough.

As shown in FIG. 1, each ramp section 102 104 may have a length of approximately 24″ and the width of approximately 72″, while each trough section 106 108 may have a length and width of approximately 72″. Furthermore, as shown the internal floor section 112 of each of the trough sections may have a length of approximately 36″. At an overall dimension of 120″×72″ with an optional safety yellow striped fabric attached to each outside rail section 114, the forklift driver has plenty of width to easily position the lift when traveling through the system. The 120″ length provides the needed length to fully submerse the circumference of a standard tire into the solution while offering a smooth and gradual travel path. The overall length of the forklift disinfecting system may also be adjusted accordingly for providing the necessary surface area to allow the full submersion of the circumference of any specific tire sizes. In some embodiments, the inside 116 and outside 114 rails of the trough sections 106 108 may be modified from a 90 degree and 2″ wide walls to a half-trapezoid shaped wall for increased wall strength. In some embodiments, the height of the system may range from 2″ to 3¼ in total height by adding a convoluted bottom section for increased product stability.

In some embodiments, the different sections of the forklift disinfecting system may be made of recycled tire rubber. In other embodiments, the trough sections may be having a full ¾″ of rubber material on their bottom sides along with half trapezoid styled walls, providing for increased durability and quality to handle the expected heavy loads. The preferred rubber material commonly known as Styrene Butadiene Rubber (SBR) is constructed using an industry standard 10/30 crumb rubber. This 10/30 crumb rubber may be mixed with industry standard buffings rubber not to exceed 25% of the total rubber mixture. Added to the rubber mixture is a polyurethane based binder (adhesive) at approximately 10% based on weight to achieve maximum strength and durability for this system.

Another material that may be used in manufacturing the sections of the modular sanitizing trough is high-density polyethylene (HDPE) or polyethylene high-density (PEHD), which is a petroleum-based polyethylene. HDPE has the advantages of its large strength to density ratio and being non-toxic, recyclable, and chemical- and corrosion-resistant. Therefore, in an example of an embodiment, the trough(s) may be made of HDPE and the ramp sections made of SBR. Other materials may also be suitable, taking into account other considerations such as cost, availability, and ease of manufacture.

In an example of an embodiment, each of the ramp sections 102 104 may weigh 115 lbs., and each trough section 106 108 may weigh 155 lbs. Accordingly, the total weight of the system may be approximately 540 lbs. Thus, a four sections system promotes the modularity of the overall system in accordance with the invention, and facilitates the ability to disassemble, transport and reassemble the system at a desired location.

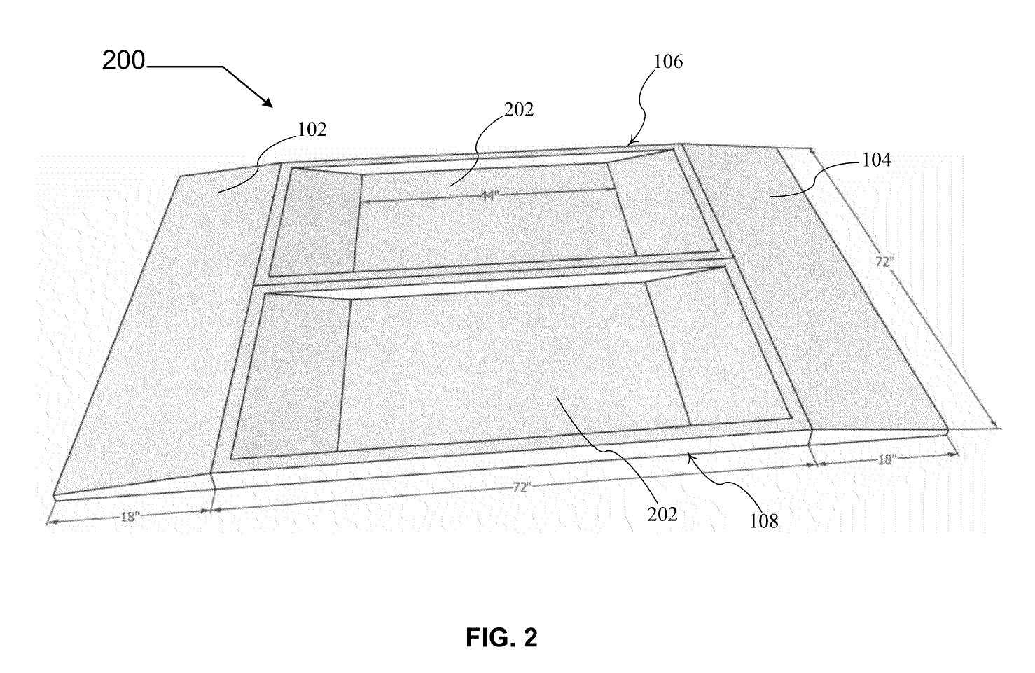

FIG. 2 shows another embodiment of the modular forklift disinfecting system 200, where the dimensions of the first 106 and second 108 trough sections may include a longer floor section 202. As shown in FIG. 2, the floor section 202 of the trough sections may be approximately 44″, thus allowing the current system to sanitize and disinfect forklifts having larger tire sizes. The length of the floor sections of a trough may change according to the size of the forklift tires typically used in the industry. It is noted that as a result of adjusting the length of the floor sections 202 from 36″ to 44″ while keeping the length of the trough sections 106 108 at 72″, the length of each of the trough ramp sections 102 104 has to also be adjusted from 18″ (in the embodiment of FIGS. 1) to 28″ (in the embodiment of FIG. 2). Thus, this results in the adjustment of the length of the first 102 and second 104 ramp sections from 24″ in FIGS. 1 to 18″ in FIG. 2.

The embodiments shown in FIGS. 1 and 2 are designed for “normal” counterbalance forklifts commonly found in warehouses and factories. It is appreciated by those skilled in the art that a modular forklift disinfecting system in accordance with the invention may have dimensions that vary from those of FIGS. 1 and 2 to accommodate an “oversized” forklift that varies from a conventional forklift To accommodate such a forklift, the width and depth of the trough may be increased, as well as the length and width of the ramps leading into and out of the trough.

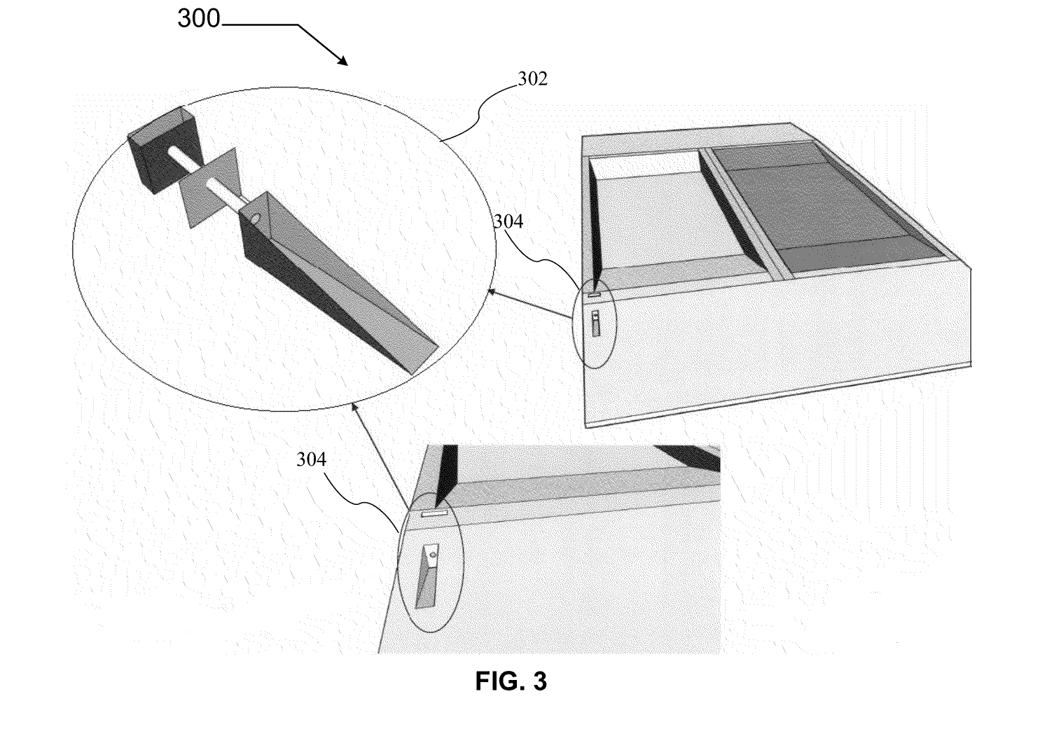

FIG. 3 illustrates yet another embodiment of the current system 300, where the feature of connecting all the sections together to minimize product migration is demonstrated. Having eight positions to connect the sections together with access from the top allows the system to be placed into a desired operating location before fastening together all of its sections. The connecting means 302 may be inserted and fastened within the depressed cutout areas 304 in the rubber or thermoplastic sections that are located below the overall height of the travel path, and thus are not exposed to the forks or the tires of the forklift. Such connecting means may include a hardware set of nuts and bolts. The bolts may be inserted into the depressed cutout areas 304 located at each of the ramp sections, while the corresponding nuts may be inserted into the depressed areas located at each of the troughs, where the nuts and bolts may be fastened. These depressed cutout areas 304 prevent the nuts and bolts from coming in contact with the wheels of the forklift. Other connecting means may include, but are not limited to other connecting hardware, for example screws, anchors, nails, etc.

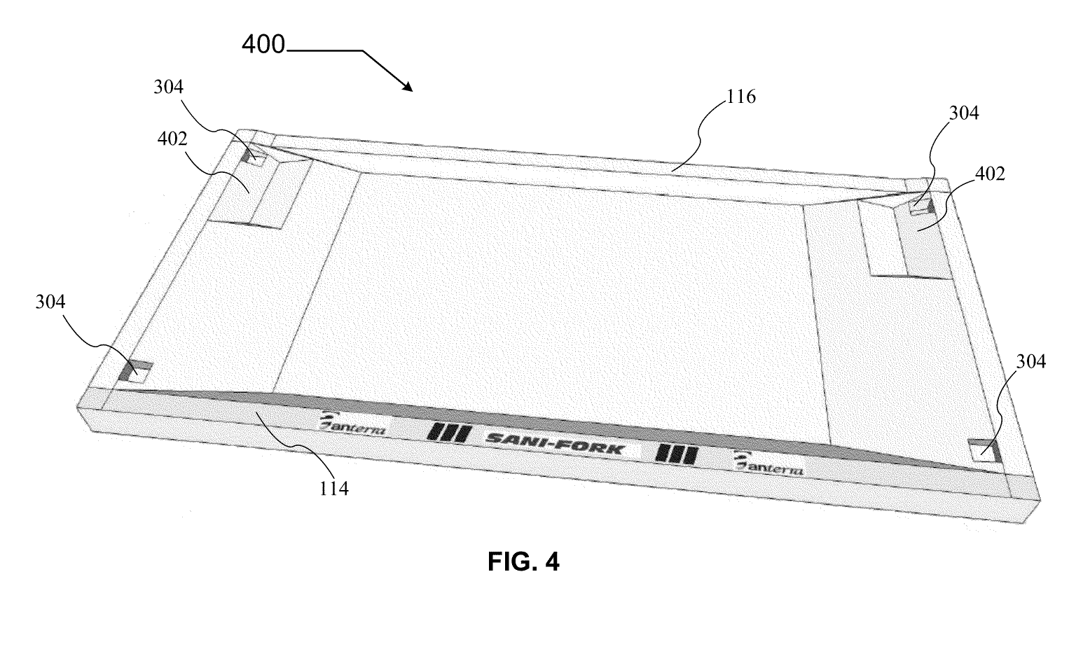

FIG. 4 shows a trough section 400 having two depressions 402. In this embodiment of a trough section, the height of the center rail 116 of the trough section 400 may be ⅜″ lower than the height of the outside rails 114. When the trough section 400 is joined with a second trough section at their respective center rails, a single trough will be formed having a center rail lower than the outside rails. These depressions or cut-out areas 402, and the reduction in the height of the center rails of a trough reduce potential damage caused by various forklifts as the forklift is traveling through the system. In some types of forklifts, the mechanism for raising and lowering the forks can cause damage to the rubber material of the trough sections when the forks are only slightly raised off ground level. Thus, the trough section as shown in FIG. 4 addresses and avoids such risks.

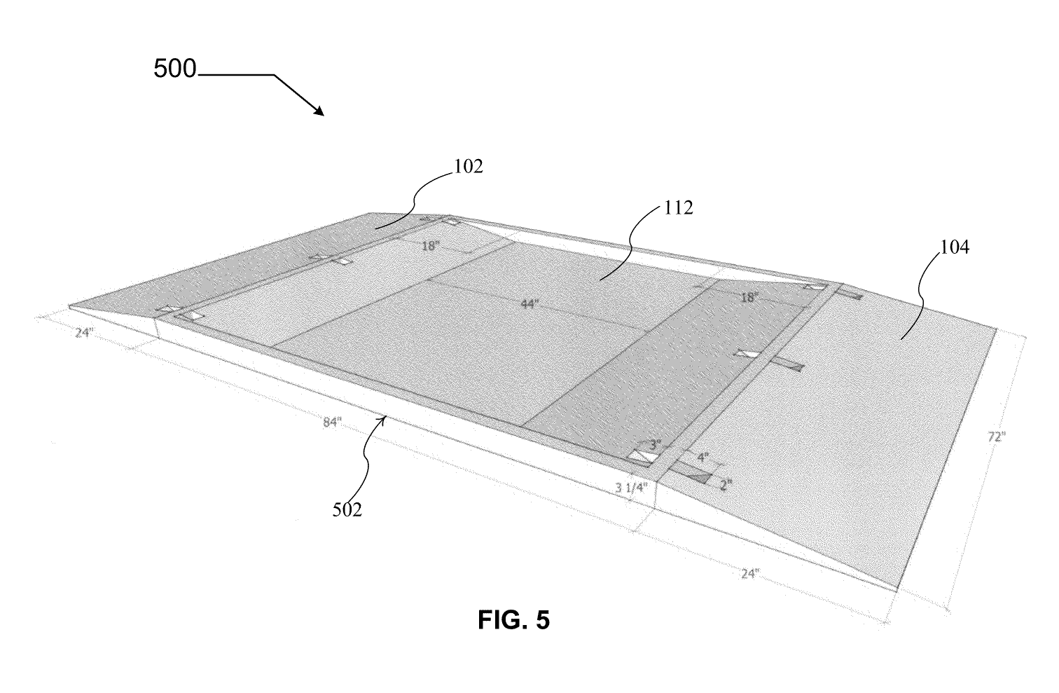

In FIG. 5, another embodiment 500 is illustrated, where the trough section 502 consists of a single trough. In these embodiments, the trough section 502 may have no central dividing rail. Thus, the sanitizing solution fills the entire floor section 112 of the trough, and may thereby be used for the purposes of disinfecting the forks and tires on both sides of the forklift. It is also noted that as illustrated in FIG. 5, the trough section 502 is shown to have a total length of approximately 84″, including a floor section 112 that has a length of approximately 44″. Thus, this length adjustment allows for the ramp sections 102 104 of the trough to be less steep, which in turn facilitates a smoother and more gradual transition of the forklift through the system. As for the depth of the trough, generally the sanitizing solution will be filled to approximately one inch below the top of the trough so as to prevent overflow of the sanitizing solution when the forklift enters the trough. Adjustments in these dimensions may be made when designing a modular forklift disinfecting system in accordance with the invention for the purposes of improving the quality of system operation.

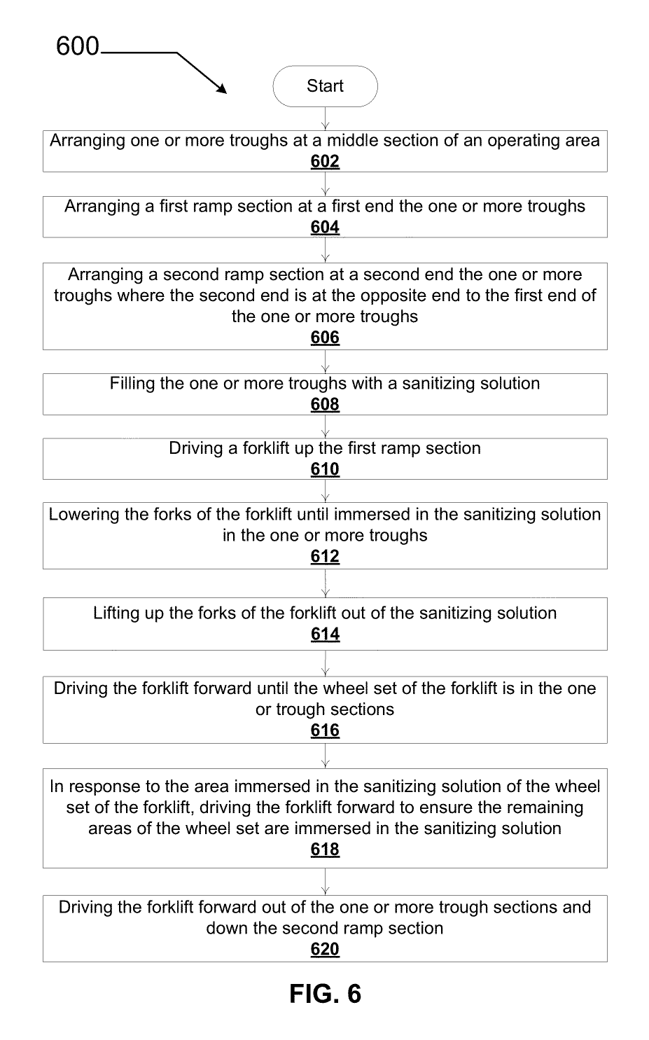

In another aspect of this invention, a method for disinfecting the tires and forks of industrial forklifts is presented. FIG. 6 illustrates the aforementioned method 600 for disinfecting the tires and forks of industrial forklifts. The process 600 will now be described by reference to FIG. 1. In step 602, one or more troughs 106 108 may be arranged at the middle section of an operating area. In step 604, a first ramp section 102 may be arranged at a first end of the one or more troughs 106 108. In step 606, a second ramp section 104 may be arranged at the second end of the one or more trough section 106 108, where the second end is at the opposite end of the first end of the one or more troughs 106 108. In step 608, the one or more trough sections 106 108 may be filled with a sanitizing solution. In step 610, a forklift may be driven up the first ramp section 102. In step 612, the forks of the forklift may be lowered until immersed in the sanitizing solution contained in the one or more trough sections 106 108. In some other embodiments, the forks may remain immersed in the sanitizing solution for thirty to sixty seconds to ensure full disinfection of the forks. In step 614, the forks may be lifted up until the forks are out of the sanitizing solution. In step 616, the forklift may be driven forward until the wheel set of the forklift is in the one or trough sections 106 108. In some embodiments, the wheel set may remain immersed in the sanitizing solution for thirty to sixty second for full disinfection of the forklift tires. In step 618, the forklift may be driven forward to ensure that the remaining areas that are not immersed in the sanitizing solution take their turn to be immersed and disinfected by the sanitizing solution. In step 620, and after the completion of the disinfecting process, the forklift may be driven out of the one or more trough sections 106 108 and down the second ramp section 104. Certain steps of method 600 may be repeated in order to completely disinfect the tires and forks of a forklift. Generally, testing has shown that a minimum of two passes is required to completely sanitize the tires of a forklift.

What is claimed is:

1. A modular forklift disinfecting system, the system comprising:
a first ramp section;
a trough section configured to contain a sanitization solution and having a first end and a second end; and
a second ramp section,
where the first ramp section is removably connected to the first end of the trough section and the second ramp section is removably connected to the second end of the trough section when the system is in an assembled state.

2. The modular forklift disinfecting system of claim 1, wherein the trough section comprises a first trough section and a second trough section.

3. The modular forklift disinfecting system of claim 2, wherein each of the first trough and the second trough comprises a first rail section and a second rail section,
wherein the trough section is formed by abutting the second rail section of the first trough section with the second rail section of the second trough section.

4. The modular forklift disinfecting system of claim 3, wherein the first rail section is higher in height than the second rail section.

5. The modular forklift disinfecting system of claim 4, wherein the first rail section has a height of approximately three and one quarter inches and the second rail section is lower in height than the first rail section by approximately three eighths of an inch.

6. The modular forklift disinfecting system of claim 3, wherein the first rail section and the second rail section each have a width of approximately two inches.

7. The modular forklift disinfecting system of claim 3, wherein the first rail section and the second rail section are half-trapezoid shaped walls.

8. The modular forklift disinfecting system of claim 1, wherein the trough section further comprises one or more wall depression areas, where a connecting means is inserted into the wall depression areas to connect the trough section to the first ramp section and the second ramp section.

9. The modular forklift disinfecting system of claim 3, wherein each of the first and second trough sections include a ramp area at each end of the trough section.

10. The modular forklift disinfecting system of claim 3, wherein each of the first and second troughs have a length of seventy two to eighty four inches and a combined width of seventy two inches.

11. The modular forklift disinfecting system of claim 1, wherein each of the first and second ramp sections has a length of eighteen to twenty four inches and a width of seventy two inches.

12. The modular forklift disinfecting system of claim 11, wherein each of the first and second troughs comprises a floor section having a length of thirty four inches.

13. The modular forklift disinfecting system of claim 1 further comprising a base plate arranged under the first ramp section, the second ramp section, and the trough section, wherein the base plate is made of Styrene Butadiene Rubber (SBR) material and having a height of three quarters of an inch.

14. The modular forklift disinfecting system of claim 1, wherein the sanitizing solution is selected from a group consisting of quaternary ammonium chloride, chlorine dioxide, peracetic acid, acid anionic, bleach, and an isopropyl alcohol-based sanitizer.

15. The modular forklift disinfecting system of claim 1, wherein the first ramp section and the second ramp section are made of SBR.

16. The modular forklift disinfecting system of claim 1, wherein the trough section is made of high-density polyethylene (HDPE).

17. A vehicle disinfecting trough comprising:
an entry ramp section;
a trough section configured to contain a sanitization solution; and
an exit ramp section.

18. A method for disinfecting forklifts, the method comprising the steps of:
arranging one or more troughs at a middle section of an operating area;
arranging a first ramp section at a first end of the one or more troughs;
arranging a second ramp section at a second end of the one or more troughs where the second end is at the opposite end to the first end of the one or more troughs;
filling the one or more troughs with a sanitizing solution;
driving a forklift up the first ramp section;
driving the forklift forward until the wheel set of the forklift is in the one or trough sections;
in response to the area immersed in the sanitizing solution of the wheel set of the forklift, driving the forklift forward to ensure the remaining areas of the wheel set are immersed in the sanitizing solution; and
driving the forklift forward out of the one or more trough and down the second ramp section.

19. The method of claim 17, the method further comprising the steps of:
lowering the forks of the forklift until immersed in the sanitizing solution in the one or more troughs; and
lifting up the forks of the forklift out of the sanitizing solution.

20. The method of claim 17, wherein the sanitizing solution is selected from a group consisting of quaternary ammonium chloride, chlorine dioxide, peracetic acid, acid anionic, bleach, and an isopropyl alcohol-based sanitizer.

21. The method of claim 17, wherein the trough section is made of high-density polyethylene (HDPE).
